(19) United States
(12) Patent Application Publication
(10) Pub. No.: US 2021/0161824 A1
(43) Pub. Date: Jun. 3, 2021

(54) ORALLY DISINTEGRATING TABLETS COMPRISING GLYCOPYRROLATE AND METHODS FOR INCREASING BIOAVAILABILITY

(71) Applicant: Balto Therapeutics, New York, NY (US)

(72) Inventors: Courtney Bond, White Plains, NY (US); Thomas Fernandez, New York, NY (US)

(21) Appl. No.: 17/049,476

(22) PCT Filed: Mar. 12, 2019
(86) PCT No.: PCT/US2019/021763

§ 371 Date: Oct. 21, 2020

(51) Int. Cl. A61K 9/20; A61K 31/4015; A61K 9/00

(52) U.S. Cl. CPC A61K 9/2072; A61K 9/0056; A61K 31/4015

(57) ABSTRACT

Orally-disintegrating porous tablets with microscopic pores comprising an effective amount of glycopyrrolate with the disintegration rate in the mouth of less than 60 seconds and an increased bioavailability. Methods for treating sialorrhea, controlling excessive production of stomach acid, controlling excessive sweating, managing stomach and/or abdominal pain, and treating drug-induced arrhythmia.

### TECHNICAL FIELD

This disclosure relates to orally-disintegrating porous glycopyrrolate tablets with an increased bioavailability and methods in which the tablets are used for administering to a patient.

### BACKGROUND

Glycopyrrolate is a quaternary ammonium compound with the following chemical name: 3-[(cyclopentylhydroxyphenylacetyl)oxy]-1,1-dimethylpyrrolidinium bromide. Its empirical formula is C₁₉H₂₈BrNO₃, its molecular weight is 398.33. Glycopyrrolate is an active ingredient in medication ROBINUL®.

Glycopyrrolate is an anticholinergic drug and is known to antagonize muscarinic symptoms. Accordingly, it can be used to control the volume and free acidity of gastric secretion. Glycopyrrolate can be also used to control excessive pharyngeal, tracheal, or bronchial secretions. Glycopyrrolate is used in the preoperative setting or during sedation as an adjunctive therapy for anesthesia. Glycopyrrolate can be also used to counter-act some of the post-operative effects.

However, not all patients tolerate well an oral glycopyrrolate formulation which cannot be easily dissolved in the patient's saliva and may have to be swollen as an intact tablet with water. In pharmacokinetic studies conducted in children and adults, glycopyrrolate has been shown to be poorly absorbed after oral administration, with a range of bioavailability from 1-20%. Furthermore, there is a considerable variation in absorption among patients. (Buck et al. 2010).

Some patients, including patients with dysphagia, cannot swallow a glycopyrrolate tablet. For these patients, oral glycopyrrolate is not recommended, yet, it is difficult to administer glycopyrrolate systemically several times a day. Furthermore, glycopyrrolate must be used with caution in patients with hepatic or renal disease. The injectable form of glycopyrrolate typically contains 0.9% benzyl alcohol as a preservative. (Buck et al. 2010). But excessive amounts of benzyl alcohol may lead to hypotension and metabolic acidosis. Accordingly, injectable forms of glycopyrrolate may not be suitable for neonates, and they should be used with caution in older infants (Buck et al. 2010).

Thus, there is a need in the art for orally-disintegrating glycopyrrolate tablets with an increased bioavailability, as such formulations may be taken without water and these formulations may be therapeutically effective with lower dosages of glycopyrrolate, which would lessen the burden on patient's liver and/or kidneys.

U.S. Pat. No. 7,091,236 discloses that the bioavailability of glycopyrrolate may be increased if a liquid formulation of glycopyrrolate is administered without food. However, these liquid formulations may not be an option for patients who have difficulties in swallowing, such as for example, as pediatric or geriatric patients. Some orally disintegrating glycopyrrolate tablets for treating sialorrhea are known from PCT/US2008/061025 and US 2008/0260823, but there is a need in the art for orally-disintegrating glycopyrrolate tablets with an increased bioavailability, especially for geriatric patients, pediatric patients, or patients with neurological disorders.

U.S. Pat. No. 5,298,261 provides methods for preparing tablets which are vacuum-dried, but there is no indication in U.S. Pat. No. 5,298,261, that this method can be used with a quaternary amine compound without affecting the bioavailability of the compound.

### SUMMARY

In one aspect, the present disclosure provides an orally-disintegrating porous tablet with microscopic pores, comprising a therapeutically effective amount of glycopyrrolate. The orally-disintegrating porous tablets disintegrate in the mouth in less than 60 seconds and they are prepared by freeze-drying a liquid suspension with a pH in the range from 4.0 to 6.5, the liquid suspension comprising at least one filler, at least one binder, at least one emulsifier, at least one disintegrating agent and water. The orally-disintegrating porous tablet has an increased bioavailability in comparison to a reference tablet comprising the same amount of glycopyrrolate. The therapeutically effective amount of glycopyrrolate may be in the range from 0.5 mg to 5 mg per one tablet. In the orally-disintegrating porous tablet of this disclosure, the disintegrating agent may comprise citric acid, succinic acid, tartic acid, or any combination thereof. In the orally-disintegrating porous tablet of this disclosure, the filler may comprise mannitol and the disintegrating agent may comprise citric acid. In the orally-disintegrating porous tablet of this disclosure, the disintegrating agent may comprise citric acid and the pH of the liquid suspension is in the range from 4.0 to 5.0. In the orally-disintegrating porous tablet of this disclosure, the filler may comprise mannitol, starch, modified starch, microcrystalline cellulose, xylitol, or any mixtures thereof. In the orally-disintegrating porous tablet of this disclosure, the binder may comprise gelatin, natural gum, synthetic gum, xanthan gum, acacia, pre-gelatinized starch, starch, methylcellulose, sodium carboxymethyl cellulose, ethyl cellulose, hydroxypropylmethyl cellulose, polyvinylpyrrolidone, polyacrylamide, or any mixtures thereof. In the orally-disintegrating porous tablet of this disclosure, the emulsifier may comprise Gelucire 44/14; Gelucire 50/13; Imwitor 91; Imwitor 308; Imwitor 380; Imwitor 742; Imwitor 780K; Imwitor 928; Imwitor 988; poloxamer 124; poloxamer 188, or any mixtures thereof. The orally-disintegrating porous tablet of this disclosure may further comprise one or more of the following: a sweetener, a flavoring agent, a coloring agent, an antibacterial agent, an anti-fungicidal agent, an absorption enhancer, a taste-masking agent, a lubricant and/or a wetting agent. In one embodiment, the orally-disintegrating porous tablet of this disclosure may further comprise sucralose and at least one flavoring agent. In another embodiment, the orally-disintegrating porous tablet of this disclosure may further comprise one or more of the following: sucralose, sucrose, glucose, aspartame, fructose, sorbitol, saccharin, cyclamate, or any mixtures thereof.

The orally-disintegrating porous tablets of this disclosure may be used for treating sialorrhea, controlling excessive production of stomach acid, controlling excessive sweating, and/or managing stomach and/or abdominal pain. The orally-disintegrating porous tablets of this disclosure may be used as a medication for a patient afflicted with at least one of the following diseases: stroke, facial paralysis, face cancer, neck cancer, esophageal cancer, mental retardation, Alzheimer's disease, Parkinson's disease, cerebral palsy, amyotrophic lateral sclerosis, peptic ulcer, intestinal ulcer, thyroid disease, or neuromuscular disease.

In further aspect, this disclosure provides a method of treating sialorrhea, controlling excessive production of stomach acid, controlling excessive sweating, and/or managing stomach and/or abdominal pain. The method comprises administering to the patient the orally-disintegrating porous tablet of this disclosure, wherein the orally-disintegrating porous tablet comprises a therapeutically effective amount of glycopyrrolate. The patient may be afflicted with at least one of the following diseases: stroke, facial paralysis, face cancer, neck cancer, esophageal cancer, mental retardation, Alzheimer's disease, Parkinson's disease, cerebral palsy, amyotrophic lateral sclerosis, peptic ulcer, intestinal ulcer, thyroid disease, or neuromuscular disease. The orally-disintegrating porous tablet of this disclosure may be administered without water. The patient may be a pediatric patient, a geriatric patient and/or the patient has dysphagia. The orally-disintegrating porous tablet may be administered in conjunction with an anesthetic agent.

Further aspects of this disclosure include methods for treating a patient, the methods comprising administering to the patient the orally-disintegrating porous tablet during induction of anesthesia, intubation or post-surgery.

Further aspects of this disclosure include methods for treating a patient for surgical or drug-induced arrhythmia, the methods comprising administering to the patient the orally-disintegrating porous tablet of this disclosure. The patient may be undergoing a treatment for drug addiction.

In further aspect, this disclosure provides a method of making the orally-disintegrating porous tablet with microscopic pores and comprising a therapeutically effective amount of glycopyrrolate, the method comprising:
mixing a water-based suspension of glycopyrrolate in the amount from 0.5 mg to 5 mg per one dosage with at least one filler, at least one binder, at least one emulsifier, and at least one disintegrating agent;
adjusting a pH of the suspension to be in the range from about 4.0 to about 6.5;
dosing the suspension by weight into blister molds;
freezing the suspension in the molds; and
drying the frozen suspension under vacuum and sublimating water, and thereby obtaining the orally-disintegrating porous tablet according to this disclosure.

In these methods, the disintegrating agent may comprise citric acid, succinic acid, tartic acid, or any combination thereof; the filler may comprise mannitol and the disintegrating agent may comprise citric acid; the disintegrating agent may comprise citric acid and the pH of the liquid suspension may be in the range from 4.0 to 5.0. The filler may comprise mannitol, starch, modified starch, microcrystalline cellulose, xylitol, or any mixtures thereof. The binder may comprise gelatin, natural gum, synthetic gum, xanthan gum, acacia, pre-gelatinized starch, starch, methylcellulose, sodium carboxymethyl cellulose, ethyl cellulose, hydroxypropylmethyl cellulose, polyvinylpyrrolidone, polyacrylamide, or any mixtures thereof. The emulsifier may comprise Gelucire 44/14; Gelucire 50/13; Imwitor 91; Imwitor 308; Imwitor 380; Imwitor 742; Imwitor 780K; Imwitor 928; Imwitor 988; poloxamer 124; poloxamer 188, or any mixtures thereof. The suspension may be further mixed with one or more of the following: a sweetener, a flavoring agent, a coloring agent, an antibacterial agent, an anti-fungicidal agent, an absorption enhancer, a taste-masking agent, a lubricant and/or a wetting agent. The suspension may be further mixed with sucralose and at least one flavoring agent. The suspension may be further mixed with one or more of the following: sucralose, sucrose, glucose, aspartame, fructose, sorbitol, saccharin, cyclamate, or any mixtures thereof.

### DETAILED DESCRIPTION

This disclosure provides an orally-disintegrating porous tablet comprising a therapeutically effective amount of glycopyrrolate. The tablet is referred in the rest of this disclosure as the orally-disintegrating glycopyrrolate tablet. The orally-disintegrating glycopyrrolate tablets of this disclosure have an increased bioavailability in comparison to other oral glycopyrrolate tablets comprising the same amount of glycopyrrolate. The orally-disintegrating glycopyrrolate tablets of this disclosure disintegrate into a suspension in less than 60 seconds in the mouth and accordingly, these tablets can be taken without water, including by patients with dysphagia or facial paralysis. Thus, the orally-disintegrating glycopyrrolate tablets of this disclosure are particularly beneficial for pediatric patients, geriatric patients and other patients who may otherwise not be able to swallow their medication with water.

Because the orally-disintegrating glycopyrrolate tablets of this disclosure have an increased bioavailability, patients can take glycopyrrolate in lower dosages. For example, a patient may be able to take 0.5 mg, 1 mg, 1.5 mg or 1.75 mg of glycopyrrolate, instead of 2 mg of glycopyrrolate per one dose. Because of the increased bioavailability, the orally-disintegrating glycopyrrolate tablets may be therapeutically effective with lower dosages of glycopyrrolate and therefore, these tablets may help with avoiding or minimizing at least some of the adverse reactions of glycopyrrolate, including xerostomia, decreased sweating, urinary hesitancy and retention, blurred vision, tachycardia, palpitations, headaches, dizziness, nausea, vomiting, nervousness, and other reactions.

The orally-disintegrating glycopyrrolate tablet of this disclosure is a solid dosage form. The orally-disintegrating glycopyrrolate tablet of this disclosure is porous and its surface comprises microscopic pores. The orally-disintegrating glycopyrrolate tablet of this disclosure disintegrates rapidly in the mouth and produces a suspension that can be easily swallowed by a patient without water. The orally-disintegrating glycopyrrolate tablet is particularly beneficial for people who may have difficulties with swallowing their medication. Such patients include patients suffering from dysphagia, pediatric patients, geriatric patients, as well as patients suffering from stroke, patients with brain injuries, patients afflicted with a thyroid disorder or a neurological disorder.

The orally-disintegrating glycopyrrolate tablet of the present disclosure may be administered to any patient whose condition or symptom may be treated or improved with a formulation comprising glycopyrrolate. These patients include cancer patients, and in particular, cancer patients afflicted with face, neck or esophageal cancer. Other patients include patients with neurological disorders or neurological disfunctions, including mental retardation, facial paralysis, excessive drooling (sialorrhea), Alzheimer's disease, Parkinson's disease, cerebral palsy or amyotrophic lateral sclerosis. The orally-disintegrating glycopyrrolate tablet of this disclosure may be beneficial to patients with gastrointestinal diseases, including peptic and/or intestinal ulcer or any other gastrointestinal conditions in which excessive production of stomach acid and/or abnormal intestinal contractions must be controlled, and/or stomach/abdominal pain must be managed.

In this disclosure, the term “to manage or control a symptom” is understood as providing a treatment which minimizes or eliminates the symptom at least for a time period, e.g. until the next dosage of glycopyrrolate must to be taken in order to control/manage the symptom. The extent of management can be determined by a patient or caregiver, for example, the patient and/or caregiver reports less drooling or less pain.

For patients who cannot swallow their medication, an intravenous injection or intramuscular administration with a syringe is typically used, however, it may be desirable to deliver glycopyrrolate orally. Furthermore, certain adverse reactions may be avoided, if glycopyrrolate is delivered orally. The orally-disintegrating glycopyrrolate tablets of this disclosure address these and other needs.

The orally-disintegrating glycopyrrolate tablets of this disclosure have an increased bioavailability in comparison to other oral tablets comprising glycopyrrolate. Glycopyrrolate must be used with caution in patients with hepatic or renal disease. Because the orally-disintegrating glycopyrrolate tablets of this disclosure have an increased bioavailability, the orally-disintegrating glycopyrrolate tablets may be therapeutically effective with lower dosages of glycopyrrolate, which may ease the burden on the liver and/or kidneys.

The increased bioavailability of the orally-disintegrating glycopyrrolate tablets of this disclosure includes both: a statistically significant increase in the rate of glycopyrrolate absorption and a statistically significant increase in the extent of glycopyrrolate absorption. The orally-disintegrating glycopyrrolate tablet of this disclosure may provide at least a 10%, and preferably at least a 20% increase in the extent of glycopyrrolate absorption in comparison to a reference oral glycopyrrolate tablet which comprises the same amount of glycopyrrolate, but which is not an orally-disintegrating porous tablet. Furthermore, the orally-disintegrating glycopyrrolate tablet of this disclosure may be taken without water, while other oral glycopyrrolate tablets are taken with substantial amounts of water, e.g. with a glass of water (approximately, 240 mL of water).

Glycopyrrolate exists in four distinct stereoisomeric forms: (R,R)-glycopyrrolate and (S,S)-glycopyrrolate, (R,S)-glycopyrrolate and (S,R)-glycopyrrolate. In some embodiments, a glycopyrrolate formulation of this disclosure comprises a mixture of at least two out of four stereoisomers: at least a mixture of (R,R)-glycopyrrolate and (S,S)-glycopyrrolate; at least a mixture of (R,R)-glycopyrrolate and (R,S)-glycopyrrolate or at least a mixture of (R,R)-glycopyrrolate and (S,R)-glycopyrrolate. In some embodiments, a glycopyrrolate formulation of this disclosure comprises a mixture of any three out of four stereoisomers. In some embodiments, a glycopyrrolate formulation comprises a mixture of all four stereoisomers. In other embodiments, the formulation comprises only one stereoisomer: (R,R)-glycopyrrolate, (S,S)-glycopyrrolate, (R,S)-glycopyrrolate or (S,R)-glycopyrrolate.

The term “therapeutically effective amount of glycopyrrolate” is to be understood broadly and means a dosage of glycopyrrolate effective to treat, ameliorate and/or at least partially improve at least some of the patient's symptoms. The therapeutically effective amount of glycopyrrolate may be adjusted by a medical practitioner, based on the patient's age, weight, severity of his/her symptoms, glycopyrrolate tolerance, and other factors.

The therapeutically effective amount of glycopyrrolate may be in the range from 0.1 mg to 10 mg per one oral tablet. Preferably, the therapeutically effective amount of glycopyrrolate may be in the range from 0.5 mg to 5 mg per one oral tablet. Most preferably, the orally-disintegrating glycopyrrolate tablets of this disclosure comprise from 0.5 mg to 3 mg of glycopyrrolate per one tablet. Preferred formulations may comprise 0.5 mg, 0.75 mg, 1 mg, 1.25 mg, 1.5 mg, 2 mg, 2.5 mg, 2.75 mg or 3 mg of glycopyrrolate per one tablet. The orally-disintegrating glycopyrrolate tablets of this disclosure may be taken from one to four times daily, e.g. two or three times daily, and the dosage and administration schedule may be further adjusted by a medical practitioner as needed. It will be appreciated that the effective dosage of glycopyrrolate is based on body weight, among other factors. For children, the dose and administration schedule must be determined by a medical doctor. The dose may be in the range from 0.01 mg of glycopyrrolate to 0.05 mg of glycopyrrolate per kilogram of body weight three times a day. A medical doctor may modify this dosage and administration schedule for each patient individually.

The glycopyrrolate tablet formulations of this disclosure disintegrate orally. These tablets dissolve or disintegrate rapidly, in less than 60 seconds in the patient's mouth. Thus, there is no need for a patient to swallow an intact tablet or chew it. Instead, the tablet rapidly disintegrates in the mouth. Furthermore, orally-disintegrating glycopyrrolate tablets of this disclosure may be taken without water or only with a minimum amount of water, e.g. one sip of water.

Rapid oral disintegration means that a tablet breaks up into smaller pieces and/or the tablet becomes at least partially dissolved, and/or forms a suspension soon after the tablet is placed in the mouth. In one preferred embodiment, the orally-disintegrating glycopyrrolate tablet formulations of the present disclosure are taken orally and they disintegrate in the mouth in less than 1 minute, less than 50 seconds, less than 40 seconds, less than 30 seconds, less than 20 seconds, or less than 10 seconds after being placed in the mouth. Preferably, the orally-disintegrating glycopyrrolate tablets of this disclosure disintegrate in the mouth within 20 to 50 seconds. The orally-disintegrating glycopyrrolate tablets of this disclosure do not require water for swallowing. Thus, the present orally-disintegrating glycopyrrolate tablets make oral glycopyrrolate formulations available for patients who have difficulties in swallowing and/or cannot swallow water. These patients may include pediatric patients, patients with neurological disorders, geriatric patients and other patients who cannot swallow water and/or fluids.

The orally-disintegrating glycopyrrolate tablets of this disclosure are porous. The orally-disintegrating glycopyrrolate tablet of this disclosure is replete with microscopic pores. A pore diameter may be in the range from 5 microns to 20 microns. In some tablets according to this disclosure, a mean pore diameter may be in the range from 10 μm to 15 μm.

The orally-disintegrating glycopyrrolate tablets of this disclosure comprise several ingredients, each of which will be now described. All percentages of ingredients in the orally-disintegrating glycopyrrolate tablets provided in this disclosure are calculated as weight-by-weight, unless stated otherwise.

The orally-disintegrating glycopyrrolate tablets of this disclosure are prepared as liquid formulations (suspensions) with water as a main solvent which may be used in combination with other co-solvents. The liquid formulations for the orally-disintegrating glycopyrrolate tablet of this disclosure may comprise from 40% by weight to 90% by weight of water. The liquid formulations are subjected to freeze-drying by which water is sublimated, and the orally-disintegrating glycopyrrolate tablets are produced.

The orally-disintegrating glycopyrrolate tablets of this disclosure may comprise from 0.1 mg to 10 mg of glycopyrrolate per one oral tablet. Preferably, the orally-disintegrating glycopyrrolate tablets of this disclosure comprise from 0.5 mg to 5 mg of glycopyrrolate per one oral tablet. Most preferably, the orally-disintegrating glycopyrrolate tablets of this disclosure comprise from 0.5 mg to 3 mg of glycopyrrolate per one tablet.

The orally-disintegrating glycopyrrolate tablets of the present disclosure may further comprise one or more excipients selected from a filler (bulking agent), a binder (gelling agent), a dispersant (emulsifier), a sweetener, a flavoring agent, a disintegrating agent, a lubricant, a colorant, a preservative and/or wetting agent.

Suitable fillers include, but are not limited to, mannitol, various starches, microcrystalline cellulose, xylitol or any mixtures thereof. Preferably, the orally-disintegrating glycopyrrolate tablets of this disclosure comprise mannitol. Typically, the filler, such as for example as mannitol, is used in an amount from 1% to 20% by weight of the liquid formulation before freeze-drying, including water, in the orally-disintegrating glycopyrrolate tablets of this disclosure. Preferably, the filler, such as for example as mannitol, is used in an amount from 1% to 10% by weight of the liquid formulation before freeze-drying, including water. Most preferably, the filler, such as for example as mannitol, is used in an amount from 1% to 5% by weight of the liquid formulation before freeze-drying, including water.

Suitable binders (gelling agents) include, but are not limited to, gelatin, natural gum, synthetic gum, xanthan gum, acacia, pre-gelatinized starch, starch, methylcellulose, sodium carboxymethyl cellulose, ethyl cellulose, hydroxypropylmethyl cellulose, polyvinylpyrrolidone, polyacrylamide, or any mixtures thereof. Preferably, the orally-disintegrating glycopyrrolate tablets of this disclosure comprise gelatin. Typically, the binder, such as for example as gelatin, is used in an amount from 1% to 20% by weight of the liquid formulation before freeze-drying, including water, for the orally-disintegrating glycopyrrolate tablets of this disclosure. Preferably, the binder, such as for example as gelatin, is used in an amount from 1% to 10% by weight of the liquid formulation before freeze-drying, including water.

Suitable emulsifiers include, but are not limited to, Gelucire 44/14; Gelucire 50/13; Imwitor 91; Imwitor 308; Imwitor 380; Imwitor 742; Imwitor 780K; Imwitor 928; Imwitor 988; poloxamer 124; poloxamer 188, or any mixtures thereof. Other suitable emulsifiers are known from U.S. Pat. No. 9,345,771 and can also used. Preferably, the orally-disintegrating glycopyrrolate tablets of this disclosure comprise poloxamer 188. Typically, the emulsifier, such as for example as poloxamer 188, is used in an amount from 0.001% to 1% by weight of the liquid formulation before freeze-drying, including water, for the orally-disintegrating glycopyrrolate tablets of this disclosure. Preferably, the emulsifier, such as for example as poloxamer 188, is used in an amount from 0.01% to 0.10% by weight of the liquid formulation before freeze-drying, including water.

Suitable sweeteners include, but are not limited to, sucralose, sucrose, glucose, aspartame, fructose, sorbitol, saccharin and its salts, cyclamate and its salts, or any mixtures thereof. Preferably, the orally-disintegrating glycopyrrolate tablets of this disclosure comprise sucralose. Typically, the sweetener, such as for example as sucralose, is used in an amount from 0.001% to 1% by weight of the liquid formulation before freeze-drying, including water, for the orally-disintegrating glycopyrrolate tablets of this disclosure. Preferably, the sweetener, such as for example as sucralose, is used in an amount from 0.1% to 1.00% by weight of the liquid formulation before freeze drying, including water.

Suitable flavoring agents include, but are not limited to, cherry flavor, black cherry flavor, mint flavor, vanilla flavor, strawberry flavor, grape flavor, cotton candy flavor or any other flavor, or mixtures thereof. Typically, the flavoring agent is used in an amount from 0.001% wt to 5% wt of the formulation total before freeze-drying, including water. Preferably, the flavoring agent is used in an amount from 0.01% wt to 1% of the formulation total, including water.

Disintegrating agents include, but are not limited to, citric acid, succinic acid, and tartic acid. Other disintegrating agents may be also used. Preferably, the orally-disintegrating glycopyrrolate tablets of this disclosure comprise citric acid. Typically, the disintegrating agent, such as for example as citric acid, is used in an amount sufficient to adjust pH of the liquid formulation for orally-disintegrating glycopyrrolate tablets to weakly acidic. A pH for the liquid formulation for the orally-disintegrating glycopyrrolate tablets may be in the range from 4.0 to 6.5, and preferably in the range from 4.0 to 5.0. Typically, from 0.01% to 1% wt of the disintegrating agent, such as for example citric acid, may be used to adjust pH of the liquid formulation as necessary.

The orally-disintegrating glycopyrrolate tablets of this disclosure may comprise some other ingredients such as one or more of the following: a coloring agent, an antibacterial agent, an anti-fungicidal agent, an absorption enhancer, a taste-masking agent such as for example, sodium bicarbonate, a lubricant and/or a wetting agent.

The orally-disintegrating glycopyrrolate tablets according to this disclosure may comprise ingredients as shown in Table 1.

TABLE 1
The Orally-Disintegrating Glycopyrrolate Tablet With Improved Bioavailability

| | | | Preferred Amount |
|---|---|---|---|
| | Amount (% wt, | | (% wt, including |
| | including water) | | water) per one |
| Ingredient | per one tablet | Preferred Species | tablet |
| Glycopyrrolate | from 0.1 mg to 10 | Glycopyrrolate | 1.5 mg to 2 mg |
| | mg | | |
| Filler | from 1% wt to 20% | Mannitol | 1% wt to 10% wt |
| | wt | | |
| Binder | from 1% wt to 20% | Gelatin | 1% wt to 10% wt |
| | wt | | |
| Emulsifier | from 0.001% wt to | Poloxamer 188 | 0.01% wt to 0.10% wt |
| | 1% wt | | |
| Sweetener | from 0.001% wt to | Sucralose | 0.01% wt to 1% wt |
| | 1% wt | | |
| Flavoring Agent | from 0.001% wt to | Black Cherry | 0.01% wt to 1% wt |
| | 5% wt | Flavor | |
| Disintegrating agent | from 0.01% wt to | Citric acid as | 0.01% wt to 1% wt |
| | 1% wt, as needed to | needed to adjust pH | |
| | adjust pH to 4.0 to | to 4.0-5.0 | |
| | 6.5 | | |
| Water (sublimed by | from 40% wt to 95% | Water (sublimed by | 50% wt to 90% wt |
| freeze-drying, the | wt | freeze-drying, the | |
| finished tablet is | | finished tablet is | |
| dried) | | dried) | |

In another aspect, the present disclosure provides the orally-disintegrating glycopyrrolate tablet which comprises some other active ingredients in addition to glycopyrrolate. These other active ingredients may include, but are not limited to, an anti-inflammatory drug and/or an analgesic.

In further aspects, the present disclosure provides methods for making the orally-disintegrating glycopyrrolate tablet of this disclosure. The orally-disintegrating glycopyrrolate tablet according to this disclosure is made by lyophilization (freeze-drying). In some freeze-drying methods, the liquid formulation comprising a therapeutically effective amount of glycopyrrolate is made with suitable excipients and water. In some embodiments, the liquid formulation comprises an effective amount of glycopyrrolate, at least one filler, at least one binder, at least one emulsifier, at least one sweetener, at least one flavoring agent, and at least one disintegrating agent. These liquid formulations can be made with water as a solvent. In further embodiments, the liquid formulation comprise ingredients listed in Table 1. In at least some of the embodiments, pH of the liquid formulation is adjusted and is in the range from about 4.0 to about 6.5. In some embodiments, pH of the suspension is adjusted and is in the range from about 4.0 to about 5.0.

Preferred liquid formulations comprise glycopyrrolate in an amount from 0.5 mg to 2 mg per one tablet, mannitol, gelatin, poloxamer 188, sucralose, at least one flavoring agent, citric acid and water. pH of the liquid formulation is adjusted and is in the range from 4.0 to 5.0.

The resulting liquid formulation, which is a suspension, may be dosed by weight into individual pre-formed blister molds in a fully automated continuous filling process. It is important that the suspension remains homogeneous during dosing. This process is regulated and monitored to ensure that each dose contains the exact amount of active ingredient—glycopyrrolate.

Once filled, the blister molds are passed through a freezing tunnel cooled by liquid nitrogen. A step of freezing may be performed at a temperature in the range from −40° C. to −80° C. This freezes water in the glycopyrrolate suspension and makes it ready for loading into low-temperature storage ahead of a freeze-drying process. As soon as a sufficient number of blister molds have been filled and frozen, water sublimation can begin by drying the blister molds. The blister molds are transferred to a freeze-dryer. The drying causes frozen water to evaporate from the solid phase to the gas phase and creates microscopic pores in a tablet formulation. During a step of primary drying, a temperature is kept below freezing. Typically, the primary drying temperature is in the range from −10° C. to −20° C. This step is completed in vacuum, such for example that the pressure is at about 0.1 mbar or lower. In a subsequent step of secondary drying, a temperature may be increased to room temperature (typically, 20° C.). This step of secondary drying may also be carried in vacuum.

When lyophilization is complete and water is sublimated, the blister molds are passed through a blister sealer, where they are sealed with aluminium foil or a suitable paper laminate. The sheets of blister molds are cut to size, and the foil perforated to facilitate opening by a patient.

In further aspects, this disclosure provides a kit comprising the orally-disintegrating porous glycopyrrolate tablets of this disclosure, prescribing information and a container, such a plastic vial, a box, and/or a sheet of blister molds. The container may be a sheet of blister molds. The prescribing information may be printed on the sheet of the blister molds.

In further aspects, this disclosure relates to methods of treatment which comprise administering to a patient the orally-disintegrating porous glycopyrrolate tablet of this disclosure, which may be administered without water and which may be further administered as an adjunctive therapy with other medications and treatments. The patients include patients in need of managing sialorrhea (excessive drooling), managing excessive sweating, preventing vomiting, managing excessive stomach acid secretion, managing excessive intestinal contractions, and/or managing stomach and/or abdominal pain. The patients include, but are not limited to, a patient with any of the following diseases or conditions: a neurological disorder, motor deficits, cerebral palsy, facial paralysis, mental retardation, Parkinson's disease, Alzheimer's disease, stroke, cancer, thyroid disease, irritable bowel syndrome, inflammatory bowel disease, Crohn's disease, gastric ulcer, stomach ulcer, or a neuromuscular disease. The patients also include any other patients for whom an anticholinergic drug can be beneficial. The patients include patients with dysphagia. The patients also include pediatric and geriatric patients.

In further aspect, the present disclosure provides a method for administering the orally-disintegrating glycopyrrolate tablet of this disclosure in conjunction with a variety of anesthetic agents, including sedatives and barbiturates. The orally-disintegrating glycopyrrolate tablet of this disclosure may be useful for patients during a preoperative procedure in order to reduce salivary, tracheobronchial and pharyngeal secretions.

In further aspect, this disclosure provides a method in which a patient is administered the orally-disintegrating porous glycopyrrolate tablet of this disclosure without water during induction of anesthesia and/or intubation.

In further aspect, this disclosure provides a method in which a patient is administered the orally-disintegrating porous glycopyrrolate tablet of this disclosure without water during a surgical procedure.

In further aspect, this disclosure provides a method in which a patient is administered the orally-disintegrating porous glycopyrrolate tablet of this disclosure without water in order to counteract surgical and/or drug-induced arrhythmia.

The drug-induced arrhythmia may be induced by narcotics (opioids) or some other controlled substances. A patient may be in need of treatment for drug overdose or he/she may be undergoing a treatment for drug addiction.

### Example 1—Comparative Pharmacokinetic and Bioavailability Analysis

The orally-disintegrating porous glycopyrrolate tablet with 2 mg of glycopyrrolate per one tablet was prepared by mixing a liquid formulation comprising glycopyrrolate, mannitol, gelatin, poloxamer 188, sucralose, a flavoring agent, and citric acid and water, as reported in Table 1. The pH of the liquid formulation with adjusted to 4.0-5.0. This suspension was then subjected to freeze-drying and tablets were produced. The resulting tablets were used as the test orally-disintegrating glycopyrrolate tablets (the test ODT) in a pharmacokinetic and bioavailability analysis as reported below.

A pharmacokinetic and bioavailability analysis was conducted for the orally-disintegrating glycopyrrolate tablet comprising 2 mg of glycopyrrolate (the test ODT). The analysis was conducted in comparison to a reference oral tablet from Par Pharmaceutical Inc which also comprised 2 mg of glycopyrrolate, referred to as the reference tablet.

The analysis included obtaining pharmacokinetic (PK) profiles of the test ODT tablet, glycopyrrolate 2 mg, and the reference tablet, glycopyrrolate 2 mg, Par Pharmaceuticals Inc. To adequately assess the potential for local irritation for subjects receiving the test ODT product, safety included a specific assessment of local safety (in the mouth), with local irritation and tolerability in the oral cavity (gross visual assessment for redness, pain and sensitivity) assessed as an adverse event (AE) of special interest. The PK profiles of glycopyrrolate were investigated after administration of a single dose of 2 mg of each of the two formulations, under fasting conditions.

This was a single dose, open-label, laboratory-blind, randomized, two period crossover study with orally administered glycopyrrolate 2 mg conducted under fasting conditions in up to 16 healthy male and female subjects at a single study center. All study participants have signed an informed consent form prior to the participation in the study.

The subjects received either the test ODT tablet or the reference tablet, according to a randomization schedule, under fasting conditions. Subjects received each tablet once.

In compliance with bioavailability/bioequivalence guidelines, the dosage in this study included a single oral dose of glycopyrrolate 2 mg (as tablets), on each of two separate occasions, under fasting conditions. The dose administered is recommended for adults (18 years and older) for the treatment of chronic severe drooling. After an overnight fast of at least 10 hours, subjects received either the reference tablet or the test ODT tablet (according to the randomization schedule). The reference tablet was swallowed whole with 240 mL water. The test ODT tablet was placed on the tongue until disintegration and then it was swallowed without water.

Safety variables included reporting of adverse events, vital signs, physical examination, oral tolerability assessment including assessment of oral mucosa, 12-lead ECG and laboratory investigations (hematology, clinical chemistry, urinalysis and pregnancy tests). Prior and concomitant medication was also recorded. Each subject was carefully monitored by the investigator for adverse events. In addition, information on adverse events was obtained from the subjects by study staff regularly questioning them, although no leading questions were asked. Data from 15 subjects who completed the study were included in the pharmacokinetic analysis.

The following Pharmacokinetic Parameters (PK) were studied:
Primary PK Parameters for Glycopyrrolate:
   Maximum observed plasma concentration (Cₘₐₓ);
   Area under the plasma concentration versus time curve, from time zero to t, where t is the time of the last quantifiable concentration (AUC₍₀₋ₜ₎); and
   Area under the plasma concentration versus time curve, with extrapolation to infinity (AUC(0-∞)).
Secondary PK Parameters for Glycopyrrolate:
   Time to maximum observed plasma concentration (tₘₐₓ);
   Terminal elimination rate constant (λ_{z}); and
   Apparent terminal elimination half-life (t_{1/2}z).

Safety variables included reporting of adverse events, vital signs, physical examination, 12-lead electrocardiogram (ECG) and laboratory investigations (hematology, clinical chemistry, urinalysis and pregnancy tests). Prior and concomitant medication was also recorded.

Table 2 provides a summary of descriptive statistics of the PK parameters.

TABLE 2
Plasma Glycopyrrolate Pharmacokinetic Parameters
(pg/mL) (Pharmacokinetic Population)

| | | | | | | |
|---|---|---|---|---|---|---|
| | | AUC₍₀₋ₜ₎ | AUC_{(0-∞)} | | | |
| | Cₘₐₓ | (h · | (h · | tₘₐₓ | λ_{z} | t_{1/2 · z} |
| Statistic | (pg/mL) | pg/mL) | pg/mL) | (h) | (1/h) | (h) |

Reference Tablet

| | | | | | | |
|---|---|---|---|---|---|---|
| n | 15 | 15 | 15 | 15 | 15 | 15 |
| Mean | 490.1 | 2513 | 2681 | — | 0.3088 | 2.365 |
| SD | 410.7 | 1484 | 1567 | — | 0.06808 | 0.6166 |
| CV % | 83.8 | 59.1 | 58.5 | — | 22.0 | 26.1 |
| Median | 433.7 | 2127 | 2213 | 3.501 | 0.3074 | 2.255 |
| Minimum | 83.40 | 645.1 | 669.6 | 1.51 | 0.1699 | 1.591 |
| Maximum | 1829 | 6724 | 7029 | 6.01 | 0.4357 | 4.079 |
| Geometric | 390.1 | 2180 | 2324 | — | 0.3013 | 2.301 |
| mean | | | | | | |
| Geometric | 77.9 | 60.2 | 60.6 | — | 24.0 | 24.0 |
| CV % | | | | | | |

Test ODT Tablet

| | | | | | | |
|---|---|---|---|---|---|---|
| n | 15 | 15 | 15 | 15 | 15 | 15 |
| Mean | 619.4 | 3165 | 3386 | — | 0.2980 | 2.494 |
| SD | 490.4 | 2349 | 2451 | — | 0.06550 | 0.8903 |
| CV % | 79.2 | 74.2 | 72.4 | — | 22.0 | 35.7 |
| Median | 426.0 | 2305 | 2452 | 3.010 | 0.2980 | 2.326 |
| Minimum | 150.0 | 1030 | 1089 | 1.01 | 0.1260 | 1.710 |
| Maximum | 2017 | 9178 | 9503 | 6.00 | 0.4054 | 5.502 |
| Geometric | 499.0 | 2631 | 2818 | — | 0.2894 | 2.395 |
| mean | | | | | | |
| Geometric | 72.7 | 64.2 | 64.9 | — | 27.5 | 27.5 |
| CV % | | | | | | |

CV = Coefficient of Variation; n = Number of subjects assessed; SD = Standard Deviation.
Reference Product (A): Glycopyrrolate 2 mg tablet, Par Pharmaceutical Inc., USA, given under fasting condition.
Test Product (B): Glycopyrrolate 2 mg orally disintegrated tablet according to the present disclosure, given under fasting condition.

The test ODT product was compared to the reference product with respect to the PK parameters for glycopyrrolate, using an analysis of variance with sequence, subject (sequence), product and period effects after logarithmic transformation of the data. Point estimates and 90% CIs for the “test/reference” geometric mean ratios of these parameters were calculated in relation to the acceptance range of 80.00% to 125.00%.

Table 3 is a summary of statistical analyses of plasma glycopyrrolate pharmacokinetic parameters.

TABLE 3
Summary of Statistical Analyses of Plasma
Glycopyrrolate Pharmacokinetic Parameters

| | | | | 90% | |
|---|---|---|---|---|---|
| | LS Mean | | % Ratio | Confidence | |
| | Test ODT | Reference | (Test/ | Interval of | Intra CV |
| Parameter (unit) | Product | Product | Reference) | Ratio | (%) |
| AUC_{(0-∞)} (h · pg/mL) | 2823.18 | 2332.10 | 121.06 | 102.26; 143.31 | 26.49 |
| AUC₍₀₋ₜ₎ (h · pg/mL) | 2638.22 | 2187.94 | 120.58 | 101.45; 143.32 | 27.14 |
| Cₘₐₓ (pg/mL) | 501.41 | 393.21 | 127.52 | 103.67; 156.86 | 32.78 |

| | | | |
|---|---|---|---|
| tₘₐₓ (h) (median) | 3.01 | 3.50 | p-value: 0.7101 |

CV = coefficient of variation;
LS = least square mean;
n: Number of subjects assessed (n = 15)
Reference Product (A): Glycopyrrolate 2 mg tablet, Par Pharmaceutical Inc., USA, given under fasting condition.
Test Product (B): Glycopyrrolate 2 mg orally disintegrated tablet according to this disclosure, given under fasting condition.

Following single dose administration of the test and reference products, maximum plasma concentrations of glycopyrrolate were reached at a median of 3.50 hours for the reference product and 3.01 hours post-dose for the test product (p-value=0.7101).

The point estimates of the “test/reference” mean ratios of the primary parameters Cₘₐₓ, AUC₍₀₋ₜ₎ and AUC_{(0-∞)} for glycopyrrolate are 127.52%, 120.58% and 121.06%, respectively.

The term “confidence interval” is abbreviated as CI. The 90% CIs for the “test/reference” mean ratios of the primary parameters Cₘₐₓ, AUC₍₀₋ₜ₎ and AUC_{(0-∞)} for glycopyrrolate are 103.67% to 156.86%, 101.45% to 143.32% and 102.26% to 143.31%, respectively.

Pharmacokinetic results and conclusions from this study are as follows. The bioavailability study was conducted to compare plasma concentrations of the test ODT product to the reference product, Glycopyrrolate 2 mg tablets. The study design was a single dose, randomized, two-way crossover study with a 3 to 14 calendar day wash-out period between doses of glycopyrrolate. Data from 15 evaluable subjects were analyzed. There was no carry-over effect of glycopyrrolate between the two treatment periods with all pre-dose values in Treatment Period 2 recorded as zero.

Dissolution of the test ODT product ranged between 11 seconds to 50 seconds with arithmetic mean dissolution time of 28 seconds.

Comparison of the bioavailability was based on the primary PK parameters of glycopyrrolate. For Cₘₐₓ the mean ratio was 127.52% with the 90% CI between 103.67% and 156.86%. The mean ratio of the AUC₍₀₋ₜ₎ was 120.58% with CI between 101.45% and 143.32%. The mean ratio of the AUC_{(0-∞)} was 121.06% with CI between 102.26% and 143.31%. The tₘₐₓ was 3.50 hours and 3.01 hours for the reference tablet and the test ODT tablet, respectively, and was not statistically different between the two products. The upper limit of the 90% CIs for all primary PK parameters were outside the pre-defined acceptance limits of 80.00% to 125.00%.

Based on the above results, it was concluded that the reference product, glycopyrrolate 2 mg tablets, and the test ODT product, glycopyrrolate 2 mg in oral disintegration tablet according to this disclosure are not equivalent with regards to both the rate and extent of absorption. The test ODT product has a higher rate and extent of absorption, leading to a conclusion that the bioavailability in the orally-disintegrating glycopyrrolate tablets according to the present disclosure is increased in comparison to a reference oral glycopyrrolate tablet.

The test ODT medication was well tolerated by all subjects (5 subjects experienced 8 AEs) during treatment with 2 mg of glycopyrrolate per treatment period. The most common AE was dry throat, experienced by 2 subjects. All the events of dry throat were considered by the investigator to be related to the IMP and of mild intensity. None of the subjects exhibited any redness, swelling or erosion/ulceration following administration of the test product. Laboratory tests, vital signs assessments, ECGs and physical examination results were compatible with expectations for a healthy male and female population.

The results of this study indicate that the test ODT medication is not equivalent to the reference tablet with respect to the rate and extent of absorption of glycopyrrolate. The test ODT tablet has an increased bioavailability, including an increase in the rate and also in the extent of absorption of glycopyrrolate.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A method of increasing the oral bioavailability of glycopyrrolate to a patient receiving oral glycopyrrolate comprising administering to the patient an orally disintegrating tablet comprising 0.5 mg to 2 mg of glycopyrrolate under fasted conditions, wherein the administration of the orally disintegrating tablet results in an increased rate and extent of absorption of glycopyrrolate without a statistically different time to achieve maximum plasma concentration as compared to administration of a reference tablet comprising an equivalent amount of glycopyrrolate.

31. The method of claim 30 wherein the orally disintegrating tablet comprises 1.7 mg of glycopyrrolate.

32. The method of claim 30 wherein the orally disintegrating tablet comprises 0.85 mg of glycopyrrolate.

33. The method of claim 30 wherein the orally disintegrating tablet comprises 1.7 mg of glycopyrrolate, a filler, a binder, and a dispersant.

34. The method of claim 30 wherein the orally disintegrating tablet comprises 0.85 mg of glycopyrrolate, a filler, a binder, and a dispersant.

35. The method of claim 30 wherein the orally disintegrating tablet comprises 1.7 mg of glycopyrrolate, a filler, a binder, and a dispersant and wherein the orally disintegrating tablet contains microscopic pores with a pore diameter in the range from 5 microns to 20 microns.

36. The method of claim 30 wherein the orally disintegrating tablet comprises 0.85 mg of glycopyrrolate, a filler, a binder, and a dispersant and wherein the orally disintegrating tablet contains microscopic pores with a pore diameter in the range from 5 microns to 20 microns.
